(19) United States
(12) Patent Application Publication
(10) Pub. No.: US 2020/0179582 A1
(43) Pub. Date: Jun. 11, 2020

(54) BEDSIDE AUTOMATED CELL ENGINEERING SYSTEM AND METHODS

(71) Applicant: LONZA WALKERSVILLE, INC., Walkersville, MD (US)

(72) Inventors: Erika MCAFEE, Pearland, TX (US); Yaling SHI, Gaithersburg, MD (US); Samatha BANDAPALLE, Monrovia, MD (US); Eytan ABRAHAM, Potomac, MD (US); Phil DENSHAM, Walkersville, MD (US); Raelyn DANIELS, Walkersville, MD (US); Nuala TRAINOR, Walkersville, MD (US); Ian GRANT, Walkersville, MD (US); Tim SMITH, Walkersville, MD (US)

(21) Appl. No.: 16/710,967

(22) Filed: Dec. 11, 2019

Related U.S. Application Data
(60) Provisional application No. 62/778,078, filed on Dec. 11, 2018.
(60) Provisional application No. 62/874,119, filed on Jul. 15, 2019.

(51) Int. Cl. A61M 1/02; A61M 1/30; C12N 15/90; C12N 13/00; C12N 15/85; C12N 5/0783; A61M 1/36

(52) U.S. Cl. CPC A61M 1/0259; A61M 1/304; C12N 15/90; A61M 1/3687; C12N 15/8509; C12N 5/0636; C12N 13/00

(57) ABSTRACT

The present disclosure provides cell therapy production systems that can suitably be used in a patient bedside setting. Such systems allow for direct removal of a patient's blood, automated processing to produce a cell therapy, and then infusion back into the patient, without the need to remove the system from the patient's bedside. Also provided herein are systems for production of cell therapies in a bedside setting.

### CROSS-REFERENCE TO RELATED APPLICATIONS OF THE INVENTION

The present application claims benefit of U.S. Provisional Patent Application No. 62/778,078, filed Dec. 11, 2018, and U.S. Provisional Patent Application No. 62/874,119, filed Jul. 15, 2019, the disclosures of each of which are incorporated by reference herein in their entireties.

### FIELD OF THE INVENTION

The present disclosure provides cell therapy production systems that can suitably be used in a patient bedside setting. Such systems allow for direct removal of a patient's blood, automated processing to produce a cell therapy, and then infusion back into the patient, without the need to remove the system from the patient's bedside. Also provided herein are systems for production of cell therapies in a bedside setting

### BACKGROUND OF THE INVENTION

As anticipation builds about accelerated clinical adoption of advanced cell therapies, more attention is turning to the underlying manufacturing strategies that will allow these therapies to benefit patients worldwide. While cell therapies hold great promise clinically, high manufacturing costs relative to reimbursement present a formidable roadblock to commercialization. Thus, the need for cost effectiveness, process efficiency and product consistency is driving efforts for automation in numerous cell therapy fields.

Automation of various processes is involved in producing cell populations for therapy. This includes integration of cell activation, transduction and expansion into a commercial manufacturing platform for the translation of these important therapies to the broad patient population.

In addition, it is highly desirable to have cell production processes be performed directly at a patient's bedside for easy and fast therapeutic applications. However, such systems must not only maintain the necessary automated processing, but also ensure sterility of the therapy and also control over the process. The present invention fulfills these needs.

### SUMMARY OF THE INVENTION

In some embodiments provided herein is a cell therapy production system, comprising: a blood withdrawal device; a cell separation device fluidly connected to the blood withdrawal device; a cell transduction apparatus fluidly connected to the cell separation filter; a cell processing apparatus fluidly connected to the cell transduction apparatus; and a cell therapy infusion device fluidly connected to the cell processing apparatus.

In further embodiments, provided herein is a cell therapy production system, comprising: a blood withdrawal device; an automated cell engineering system, including: an enclosable housing; a cell separation device contained within the enclosable housing and fluidly connected to the blood withdrawal device; a cell transduction apparatus contained within the enclosable housing and fluidly connected to the cell separation filter; and a cassette contained within the enclosable housing, the cassette comprising a cell culture chamber fluidly connected to the cell transduction apparatus; and a cell therapy infusion device fluidly connected to the cell culture chamber.

Also provided herein is a method for preparing a cell therapy product, the method comprising: withdrawing a blood sample from a patient; passing the blood sample through a cell separation device to remove a target cell population from the blood sample; transducing the target cell population with a vector to produce a transduced cell culture; optionally expanding the transduced cell culture; harvesting the cell culture; and infusing the harvested cell culture into the patient.

### BRIEF DESCRIPTION OF THE FIGURES

FIG. 1 shows a cell therapy production system in accordance with embodiments hereof.

FIG. 2 shows exemplary components of a call therapy production system in accordance with embodiments hereof.

FIGS. 3A-3B show an automated cell engineering system in accordance with embodiments hereof.

FIG. 4 shows a cartridge for use in embodiments hereof.

FIG. 5 shows a flowpath of a cell therapy production system as described herein.

### DETAILED DESCRIPTION OF THE INVENTION

It should be appreciated that the particular implementations shown and described herein are examples and are not intended to otherwise limit the scope of the application in any way.

The published patents, patent applications, websites, company names, and scientific literature referred to herein are hereby incorporated by reference in their entirety to the same extent as if each was specifically and individually indicated to be incorporated by reference. Any conflict between any reference cited herein and the specific teachings of this specification shall be resolved in favor of the latter. Likewise, any conflict between an art-understood definition of a word or phrase and a definition of the word or phrase as specifically taught in this specification shall be resolved in favor of the latter.

As used in this specification, the singular forms “a,” “an” and “the” specifically also encompass the plural forms of the terms to which they refer, unless the content clearly dictates otherwise. The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%.

Technical and scientific terms used herein have the meaning commonly understood by one of skill in the art to which the present application pertains, unless otherwise defined. Reference is made herein to various methodologies and materials known to those of skill in the art.

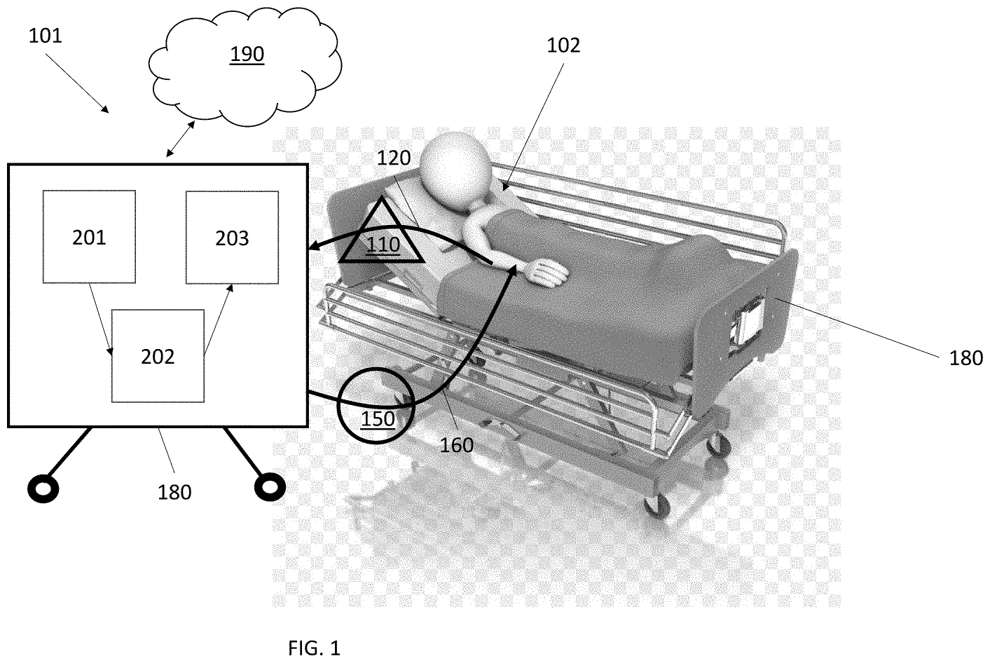

In embodiments, provided herein are cell therapy production systems. FIG. 1 shows a schematic illustrating a cell therapy production system 101, as well as a patient 102. Cell therapy production system 101 illustrates via block diagram, exemplary components for carrying out or performing the production of a cell therapy. Activities include cell separation 201 from a sample collected from a patient. Cell separation 201 is suitably followed by cell transduction 202, and then by cell processing 203.

As used herein, a “cell therapy” or “cellular therapy” refers to a treatment in which cellular material is injected, infused, grafted or implanted into a patient. Cell therapies suitably include intact, living cells, and in embodiments, include cells taken from the patient's own body.

In embodiments, the cell production systems 101 described herein are designed for use with a single patient at a time. That is, they are designed and utilized in the context of a cellular therapy being prepared directly onsite, for example at a hospital, treatment facility, clinic, or home, such that the patient can be treated at his or her bedside. It should be understood that “bedside” as used herein simply refers to a location that is convenient and near to a patient, and can include directly next to a patient's bed (stretcher, chair, etc.), but can also be simply within the same room or building as the patient, but without requiring removal of a sample from the patient, transport to another location (even within the same building) and then processing. The cell production systems 101 described herein provide direct interaction between the patient and the cell production system to minimize contamination, minimize transport of bodily fluids, and minimize patient mix-up or incorrect labeling, etc.

Suitably, as illustrated in FIG. 1, cell production systems 101 described herein include a blood withdrawal device 110 for removal of blood 120 from a patient 102. Exemplary blood withdrawal devices include various pumps or suction devices, coupled with tubing and needles for insertion into a patient. In embodiments, blood withdrawal device 110 can be an apheresis device for collection of a patient's blood.

Cell production systems 101 suitably also include a cell separation device for cell separation 201 fluidly connected to the blood withdrawal device 110. Exemplary cell separation devices include various magnetic separation devices that can include the use of magnetic beads (e.g., DYNABEADS), as well as filtration (including column filtration devices and filtration media), separation media, centrifugation, etc. In embodiments, cell separation can also be carried out in the blood withdrawal device 110, for example in the case of an apheresis device that separate out different components of blood via centrifugation.

As used herein, “fluidly connected” means that one or more components of a system, are connected via a suitable element that allows for fluids (including gasses and liquids) to pass between the components without leaking or losing volume. Exemplary fluid connections include various tubing, channels and connections known in the art, such as silicone or rubber tubing, luer lock connections, etc. It should be understood that components that are fluidly connected can also include additional elements between each of the components, while still maintaining a fluid connection. That is, fluidly connected components can include additional elements, such that a fluid passing between the components can also pass through these additional elements, but is not required to do so.

Cell production systems 101 also further include a cell transduction device for cell transduction 202 fluidly connected to the cell separation device. As used herein, “transduction” or “transducing” means the introduction of an exogenous nucleic acid molecule, including a vector, into a cell. A “transduced” cell comprises an exogenous nucleic acid molecule inside the cell and induces a phenotypic change in the cell. The transduced nucleic acid molecule can be integrated into the host cell's genomic DNA and/or can be maintained by the cell, temporarily or for a prolonged period of time, extra-chromosomally. Host cells or organisms that express exogenous nucleic acid molecules or fragments are referred to as “recombinant,” “transduced,” “transfected,” or “transgenic” organisms. A number of transduction and transfection techniques are generally known in the art. See, e.g., Graham et al., Virology, 52:456 (1973); Sambrook et al., Molecular Cloning, a laboratory manual, Cold Spring Harbor Laboratories, New York (1989); Davis et al., Basic Methods in Molecular Biology, Elsevier (1986); and Chu et al., Gene 13:197 (1981). Transduction can include the use of a transfection system such as a liposome, lipid-based, or polymer-based system, and can also include the use of mechanical transfection such as gene guns, electroporation, etc.

Cell production system 101 also suitably further includes a cell processing apparatus fluidly connected to the cell transduction apparatus for cell processing 203. As used herein a “cell processing apparatus” refers to an enclosed, suitably sterile and automated system, for processing a cell therapy following transduction and prior to infusion back into a patient. Exemplary activities that can be carried out by a cell processing apparatus include, for example, filtering, washing, diluting and/or formulating. “Formulating” as used herein refers to the addition of, for example, a media or solution to assist with buffering or stabilizing a cell therapy, as well as the addition of preservatives, pH modifiers, osmolality modifiers, various salts and excipients, etc.

Cell production system 101 also suitably includes a cell therapy infusion device 150 for returning the cell therapy 160 to the patient 102 fluidly connected to the cell processing apparatus. Cell therapy infusion device suitably includes one or more pumps, as well as fluid tubing, etc., for transporting the cell therapy from the cell processing apparatus to the patient, and for example via a needle, injecting or infusing (i.e., slow injection over time) the cell therapy to the patient 102. Blood withdrawal device 110 and cell therapy infusion device 150 can also be the same device, for example an apheresis device.

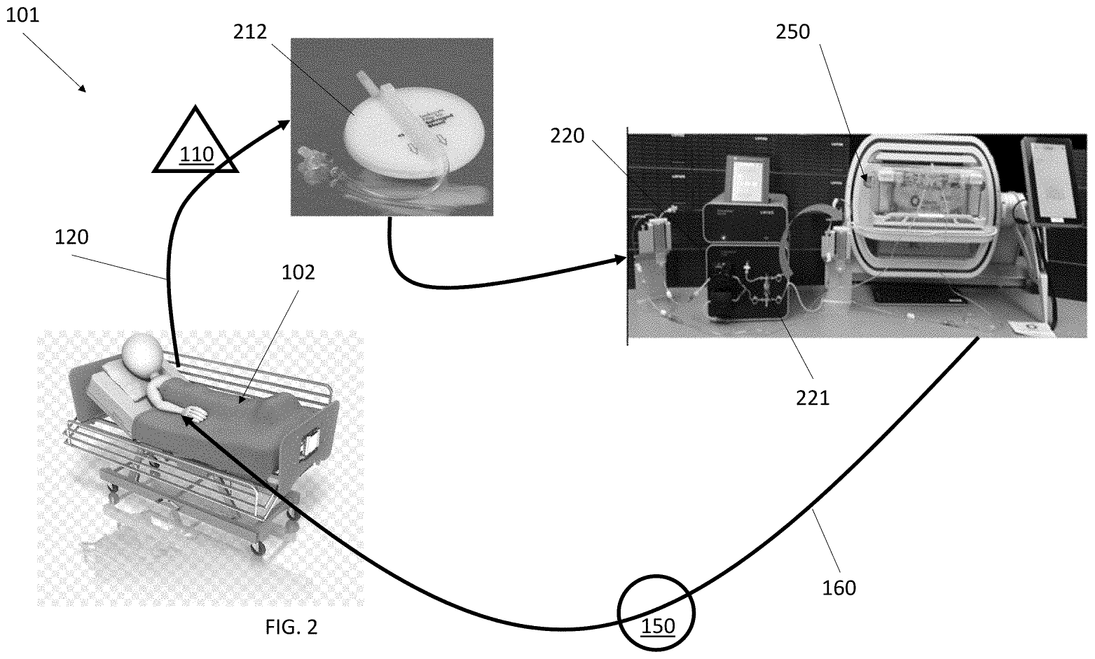

FIG. 2 shows exemplary components of a cell therapy production system 101. For example, the cell separation device utilized in the cell therapy production system can be a cell separation filter 212. In exemplary embodiments, cell separation filter 212 includes a matrix which captures a cell population, suitably target cells. Suitable matrix materials include various porous media that has been treated with a gas plasma. The porous media can be a natural or synthetic fiber or woven material, or a sintered powder material. Exemplary matrix materials include those disclosed in, for example, U.S. Pat. Nos. 4,701,267, 4,936,998, 4,880,548, 4,923,620, 4,925,572, and 5,679,264, the disclosures of each of which are incorporated by reference herein in their entireties. As used herein a “target cell population” or “target cells” refers to a desired sub-set of cells that is to be separated from a larger cell population, including from debris or other contaminants, such that the remaining target cell population is largely free of other cell types. Exemplary target cell populations include immune cells, cancer cells, etc.

Exemplary cell separation filters suitably include a matrix that allows for the capture of immune cells, that is the matrix retains immune cells on or within the matrix. As used herein, “immune cells” includes basophils, eosinophils, neutrophils, leukocytes, etc., and include cells such as mast T-cells, dendritic cells, naturally killer cells, B cell, T-cells, etc. Suitably, the target cell population is a T-cell population, which can be used for the production of CAR T-cells as described herein.

As described herein, the cell separation filters are suitably used to separate immune cells from a cellular sample, including a whole blood cell sample or a leukophoresis sample (sample in which white blood cells are separated from whole blood), that is withdrawn from a patient. Exemplary methods and cell separation filters for removal of target cells from whole blood are described in U.S. Provisional Patent Application No. 62/778,078, filed Dec. 11, 2018, the disclosure of which is incorporated by reference herein in its entirety.

In embodiments, as illustrated in FIG. 2, the cell transduction apparatus suitably is an electroporation unit 220, which is fluidly connected to the output of the cell separation device, e.g., cell separation filter 212. Electroporation unit 220 suitably includes an electroporation cartridge 221, which holds the cells during the electroporation process. Following the electroporation process, the transduced cells are transferred to cell processing apparatus 250. In embodiments, two optional reservoirs can also be used to hold the cells prior to and after electroporation, to help in the transfer between the cell processing apparatus 250 and the electroporation unit 220 as a result of different pump speeds, required pressures and flow rates. However, such reservoirs can be removed and the cells transferred directly from electroporation unit 220 to cell processing apparatus 250.

In exemplary embodiments, as shown in FIG. 2, electroporation unit 220 can be located separately from cell processing apparatus 250. In such embodiments, the transducing comprises transferring via a first sterile, closed connection (e.g., connection tubing), the target cell population from the cell separation device (e.g., cell separation filter 212) to the electroporation unit 220, electroporating the target cell population with a vector, to produce a transduced cell culture, and transferring via a second sterile, closed connection, the transduced cell culture to the cell processing apparatus 250.

It should also be understood that multiple, separate cell separation devices can be connected to a single electroporation unit, and run in appropriate order such that cells are transferred from the cell separation devices, to the electroporation unit, and then to the cell processing apparatus.

Electroporation unit 220 enables transfection of cells traditionally known to have low transfection efficiency via electroporation and other non-viral methods, including primary cells, stem cells, neurons, and resting or non-proliferating cells. The system includes an electroporation unit, electroporation solutions, electroporation Cartridges and optimized electroporation protocols. The electroporation unit is suitably comprised of a Core Unit and 1-3 additional functional add-on units addressing different needs. For example, the electroporation unit can be used to transfect varying cell numbers in 20 μL-100 μL and 1×10⁷ to 1×10⁹ in 1 mL-20 mL volume

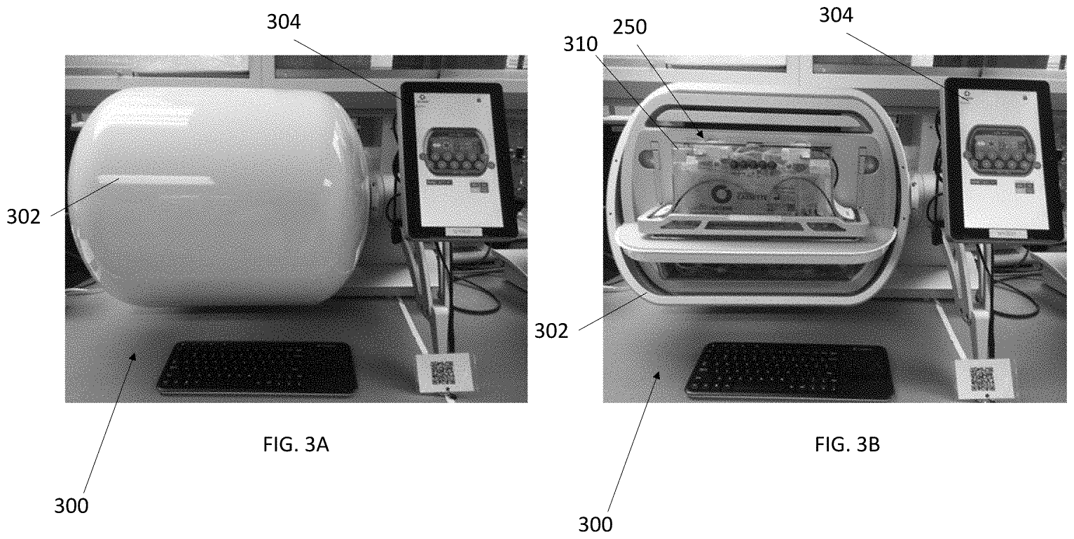

In exemplary embodiments, the cell separation device (e.g., cell separation filter 212), the cell transduction apparatus (e.g., electroporation unit 220), and cell processing apparatus 250 are suitably contained within an automated cell engineering system 300, as illustrated in FIGS. 3A-3B. Automated cell engineering systems 300 suitably include a cassette 310, in which the various processes of the cell processing apparatus 250 (e.g., washing, filtering, diluting, formulation, etc.) can be carried out in an enclosed, automated system that allows for production of various cellular samples and populations. Such processes can also include activating, transducing, expanding, concentrating, and collecting/harvesting steps.

As described herein, the cassettes and methods are suitably utilized and carried out in a fully enclosed automated cell engineering system 300 (see FIGS. 3A, 3B), suitably having instructions thereon for performing steps such as, activating, transducing, expanding, concentrating, and harvesting. Cell engineering systems for automated production of, for example genetically modified immune cells, including CAR T-cells, are described in U.S. Published Patent Application No. 2019/0169572 (the disclosure of which is incorporated by reference herein in its entirety), and are also called automated cell engineering system, COCOON, or COCOON system herein.

For example, a user can provide an automated cell engineering system pre-filled with a cell culture and reagents (e.g., an activation reagent, a vector, cell culture media, nutrients, selection reagent, and the like) and parameters for the cell production (e.g., starting number of cells, type of media, type of activation reagent, type of vector, number of cells or doses to be produced, and the like), the automated cell engineering system is able to carry out the various automated methods, including methods of producing genetically modified immune cell cultures, including CAR T-cells, without further input from the user. In some embodiments, the fully enclosed automated cell engineering system minimizes contamination of the cell cultures by reducing exposure of the cell culture to non-sterile environments. In additional embodiments, the fully enclosed automated cell engineering system minimizes contamination of the cell cultures by reducing user handling of the cells.

As described herein, the automated cell engineering systems 300 suitably include a cassette 310. As used herein a “cassette” refers to a largely self-contained, removable and replaceable element of an automated cell engineering system that includes one or more chambers for carrying out the various elements of the methods described herein, and suitably also includes one or more of a cell media, an activation reagent, a wash media, etc.

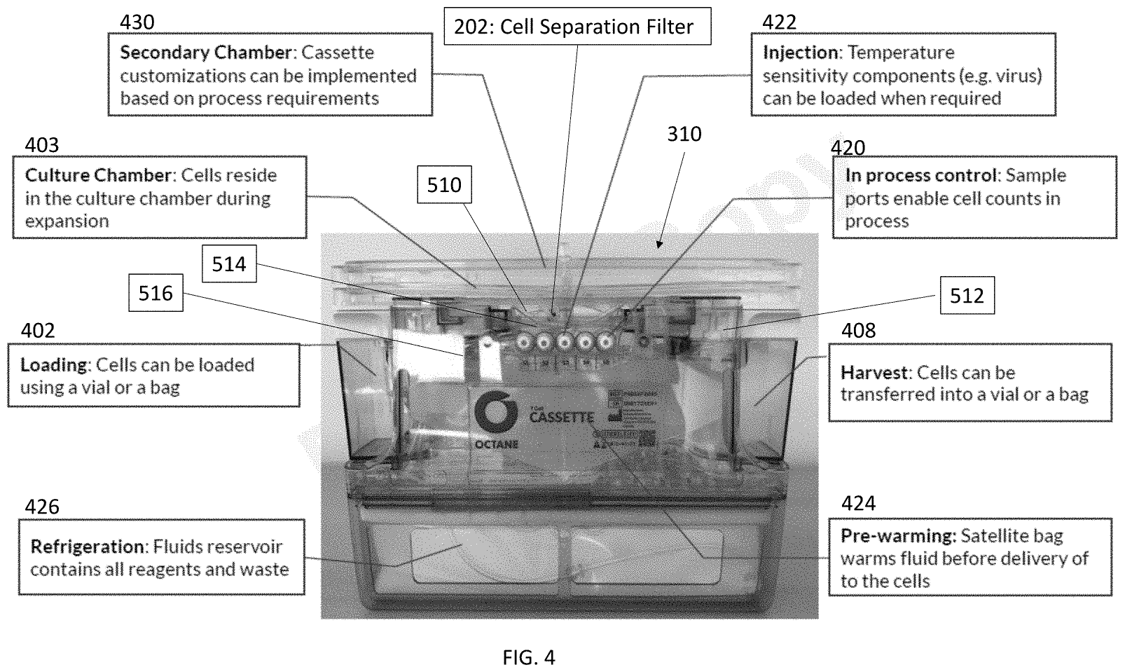

FIG. 4 shows an exemplary cassette 310 for use in an automated cell engineering system. In embodiments, cassette 310 includes a cellular sample input 402. Cellular sample input 402 is shown in FIG. 4 as a vial or chamber in which a cellular sample can be placed prior to introduction or loading into cassette 310. In other embodiments, cellular sample input 402 can simply be a sterile-locking tubing (for example a luer lock tubing connection or the like) to which a syringe or a cell-containing bag, such as a blood bag, can be connected. In suitable embodiments, cellular sample input 402 is directly connected to blood withdrawal device 110, or the output of the cell separation device (e.g., cell separation filter 212) so that a blood sample (either separation or unseparated), can be directly input into the cassette 310.

In embodiments, cell processing apparatus 250 suitably includes a cell culture chamber 403, which in embodiments, can be part of cassette 310.

As described herein, suitably cassette 310 can include cell separation filter 212, located within the cassette, and fluidly connected to cellular sample input 402. Cassette 310 suitably further includes the cell culture chamber 403 fluidly connected to the cell separation filter 212. Examples of the characteristics and uses of cell culture chamber 403 are described herein.

In embodiments, cassette 310 further includes one or more fluidics pathways connected to the cell culture chamber (see inside cassette 310 in FIG. 4). Also included in cassette 310 is a cellular sample output 408 fluidly connected to cell culture chamber. As described herein, cellular sample output 408 is utilized to harvest the cells following the various automated procedures for either further processing, storage, or suitably for use and infusion directly into a patient via cell therapy infusion device 150. Examples of fluidics pathways include various tubing, channels, capillaries, microfluidics elements, etc., that provide nutrients, solutions, etc., to the elements of the cassette, as described herein.

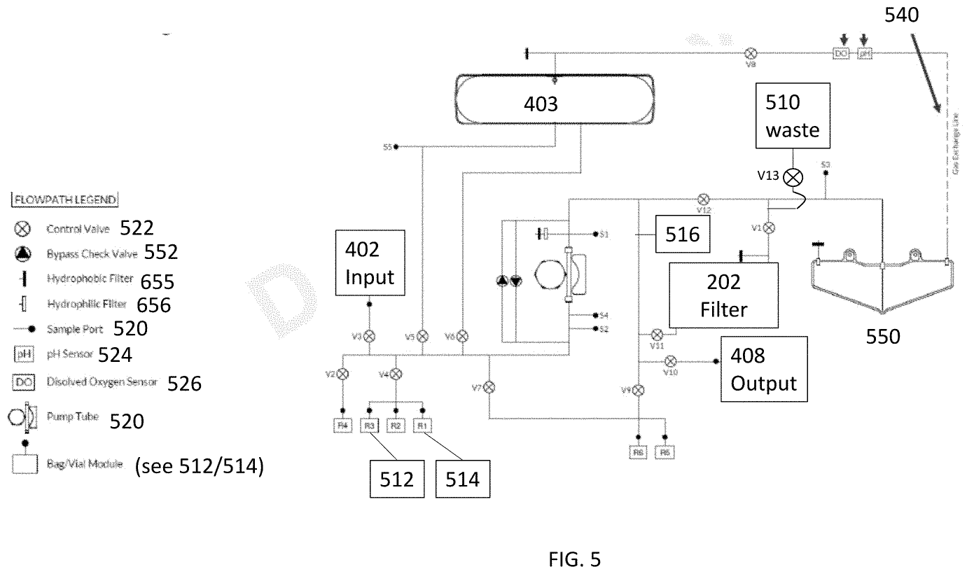

As described herein, the fluidics pathways, which can include various tubing elements, suitably provide recirculation, removal of waste and homogenous gas exchange and distribution of nutrients to various parts of the cassette, including the cell culture chamber without disturbing cells within the cell culture chamber. Cassette 310 also further includes one or more pumps 520 and related tubing, including peristaltic pumps, for driving fluid through the cassette, as described herein, as well as one or more valves 522, for controlling the flow through the various fluidic pathways (see FIG. 5 for exemplary locations within flowpath).

In exemplary embodiments, as shown in FIG. 4, cell culture chamber 403 is flat and non-flexible chamber (i.e., made of a substantially non-flexible material such as a plastic) that does not readily bend or flex. The use of a non-flexible chamber allows the cells to be maintained in a substantially undisturbed state. As shown in FIG. 4, cell culture chamber 403 is oriented so as to allow a cell culture to spread across the bottom of the cell culture chamber. As shown in FIG. 4, cell culture chamber 403 is suitably maintained in a position that is parallel with the floor or table, maintaining the cell culture in an undisturbed state, allowing the cell culture to spread across a large area of the bottom of the cell culture chamber. In embodiments, the overall thickness of cell culture chamber 403 (i.e., the chamber height) is low, on the order of about 0.5 cm to about 5 cm. Suitably, the cell culture chamber has a volume of between about 0.50 ml and about 300 ml, more suitably between about 50 ml and about 200 ml, or the cell culture chamber has a volume of about 180 ml. The use of a low chamber height (less than 5 cm, suitably less than 4 cm, less than 3 cm, or less then 2 cm) allows for effective media and gas exchange in close proximity to the cells. Ports are configured to allow mixing via recirculation of the fluid without disturbing the cells. Larger height static vessels can produce concentration gradients, causing the area near the cells to be limited in oxygen and fresh nutrients. Through controlled flow dynamics, media exchanges can be performed without cell disturbance. Media can be removed from the additional chambers (no cells present) without risk of cell loss. In other embodiments, cell culture chamber 403 is a bag or hard chamber.

As described herein, in exemplary embodiments the cassette is pre-filled with one or more of a cell culture, a culture media, a cell wash media, a back flush media, an activation reagent, a dilution media, a formulation media, a buffer, one or more excipients, and/or a vector, including any combination of these. In further embodiments, these various elements can be added later via suitable injection ports, etc. In exemplary embodiments the back flush media suitably contains an anticoagulant, such as ethylenediaminetetraacetic acid (EDTA), to reduce clumping of the target cell population that is transferred from the separation filter. In embodiments, the cassette includes elements for formulating a cell therapy, including various excipients, dilution buffers, salts, pH modifying agents, osmolality modifying agents, etc., to be used by the cell processing apparatus to prepare the cell therapy for infusion directly from the apparatus into a patient.

As described herein, in embodiments, the cassettes suitably further include one or more of a pH sensor 524, a glucose sensor (not shown), an oxygen sensor 526, a carbon dioxide sensor (not shown), a lactic acid sensor/monitor (not shown), and/or an optical density sensor (not shown). See FIG. 5 for exemplary positions within the flowpath. The cassettes can also include one or more sampling ports and/or injection ports. Sampling ports and injection ports can include an access port for connecting the cartridge to an external device, such as an electroporation unit or an additional media source.

In embodiments, cassette 310 suitably includes a low temperature chamber, which can include a refrigeration area 426 suitably for storage of a cell culture media, as well as a high temperature chamber, suitably for carrying out activation, transduction, transfection and/or expansion of a cell culture. Suitably, the high temperature chamber is separated from the low temperature chamber by a thermal barrier. As used herein “low temperature chamber” refers to a chamber, suitably maintained below room temperature, and more suitably from about 4° C. to about 8° C., for maintenance of cell media, etc., at a refrigerated temperature. The low temperature chamber can include a bag or other holder for media, including about 1 L, about 2 L, about 3 L, about 4 L, or about 5 L of fluid. Additional media bags or other fluid sources can be connected externally to the cassette, and connected to the cassette via an access port.

As used herein “high temperature chamber” refers to chamber, suitably maintained above room temperature, and more suitably maintained at a temperature to allow for cell proliferation and growth, i.e., between about 35-40° C., and more suitably about 37° C. In embodiments, high temperature chamber suitably includes cell culture chamber 206 (also called proliferation chamber or cell proliferation chamber throughout).

As shown in FIGS. 3A and 3B, automated cell engineering system 300 suitably includes an enclosable housing 302, and cell processing apparatus 250 including cassette 310, contained within the enclosable housing. As used herein, “enclosable housing” refers to a structure than can be opened and closed, and within which cassette 310 as described herein, can be placed and integrated with various components such as fluid supply lines, gas supply lines, power, cooling connections, heating connections, etc. As shown in FIGS. 3A and 3B, enclosable housing can be opened (FIG. 3B) to allow insertion of the cassette, and closed (FIG. 3A) to maintain a closed, sealed environment to allow the various automated processes described herein to take place utilizing the cassette.

FIGS. 3A and 3B show the automated cell engineering system 300 with cassette 310 positioned inside (enclosable housing 302 of automated cell engineering system 300 opened in FIG. 3B). Also shown is an exemplary user interface 304, which can include a bar code reader, and the ability to receive using inputs by touch pad or other similar device.

The automated cell engineering systems and cassettes described herein suitably have three relevant volumes, the cell culture chamber volume, the working volume, and the total volume. Suitably, the working volume used in the cassette ranges from 180 mL to 460 mL based on the process step, and can be increased up to about 500 mL, about 600 mL, about 700 mL, about 800 mL, about 900 mL or about 1 L. In embodiments, the cassette can readily achieve 4*10⁹ cells-10*10⁹ cells. The cell concentration during the process varies from 0.3*10⁶ cells/ml to approximately 10*10⁶ cells/ml. The cells are located in the cell culture chamber, but media is continuously recirculated through additional chambers (e.g., crossflow reservoir and satellite volume) to increase the working volume, as described herein.

Fluidics pathways, including gas exchange lines, may be made from a gas-permeable material such as, e.g., silicone. In some embodiments, the automated cell engineering system recirculates oxygen throughout the substantially non-yielding chamber during the cell production methods. Thus, in some embodiments, the oxygen level of a cell culture in the automated cell engineering system is higher than the oxygen level of a cell culture in a flexible, gas-permeable bag. Higher oxygen levels may be important in the cell culture expansion step, as increased oxygen levels may support increased cell growth and proliferation.

In embodiments, the methods and cartridges described herein are utilized the COCOON platform (Octane Biotech (Kingston, ON)), which integrates multiple unit operations in a single turnkey platform. Multiple cell protocols are provided with very specific cell processing objectives. To provide efficient and effective automation translation, the methods described utilize the concept of application-specific/sponsor-specific disposable cassettes that combine multiple unit operations—all focused on the core requirements of the final cell therapy product. Multiple automated cell engineering systems 300 can be integrated together into a large, multi-unit operation for production of large volumes of cells or multiple different cellular samples for individual patients (see FIG. 4).

As shown in FIGS. 3A-3B, automated cell engineering system 300 also further includes a user interface 304 for receiving input from a user. User interface 304 can be a touch pad, tablet, keyboard, computer terminal, or other suitable interface, that allows a user to input desired controls and criteria to the automated cell engineering system to control the automated processes and flowpath. Suitably, the user interface is coupled to a computer control system to provide instructions to the automated cell engineering system, and to control the overall activities of the automated cell engineering system. Such instructions can include when to open and close various valves, when to provide media or cell populations, when to increase or decrease a temperature, etc.

As described herein and illustrated in FIG. 1, suitably the cell therapy production system 101 described herein is portable. As used herein “portable” refers to the ability to locate and then re-locate, or move, cell therapy production system 101 between one or more locations where the production of a cell therapy is desired. For example, cell therapy production system 101 can be placed on a cart 180, table, wheeled platform, or other structure that allows the system to be moved from place to place easily. For example, the system can be readily moved from one patient to another patient, or one patient's bedside to another patient's bedside, within a hospital, clinic, or other setting, to allow for the system to be utilized by multiple patients quickly and easily one after the other. It should also be understood that the systems described herein can be stationary and the patient(s) come to the systems. That is each patient is moved to a single location where the system is placed, the therapy is conducted, and then the patient is removed and another patient is treated.

In embodiments, the systems described herein further include a bed 190, such that the blood withdrawal device 110 and the cell therapy infusion device 150 are collocated with the bed. In such embodiments, the remainder of the components of the system described herein (e.g., cell separation device 201, cell transduction apparatus 202 and cell processing apparatus 203), can be brought into the room with such a bed 190, or the bed 190 can be brought into a room with the remaining components of the system, and the system and the blood withdrawal and infusion devices connected to the system 101. As used here “collocated” means that the components described herein are suitably located within the same room, suitably connected to each other, but can also include in the same building, hospital etc. For example, the blood withdrawal device and the cell therapy infusion device can be plumbed in to a wall or other structure, and a bed collocated in the same room.

In additional embodiments, as illustrated in FIG. 1, the cell therapy production systems 101 described herein further include an automated process control system (APCS) 190, which is configured to control the system. Exemplary automated process control systems are described in U.S. Provisional Patent Application No. 62/874,119, filed Jul. 15, 2019, the disclosure of which is incorporated by reference herein in its entirety.

Automated process control systems, as discussed herein, may interact with, receive inputs from, provide inputs to, and otherwise provide all aspects of control of one or more cell therapy production systems. In embodiments, a network environment may be used to monitor the cell therapy production systems. The network environment may include one or more automated process control system (APCS) 190 in communication with one or more cell therapy production systems 101, one or more data retention systems, one or more clients, via one or more networks. The cell therapy production systems may be in a single location (e.g., one hospital or one clinic) or may be located through several hospitals across a city, a state, a country or the world.

Data and information stored by cell therapy production system 101 may include the following information. As used herein, “cell therapy production system data” refers to any and all data that may be recorded and stored on or in a memory of a cell therapy production system 101. Cell therapy production system data may be stored in any suitable data format, and may be sortable by production batch, production date, or any other suitable parameter. “Process information,” as used herein, refers to information about variables and parameters of cell culture processing, including, for example, one or more of temperature information, pH information, glucose concentration information, oxygen concentration information, component or patient identification information and optical density information, from the cell therapy production system. Production information, as used herein, may refer to information about cell culture growth, including one or more of number of cells, cell characteristics, % transformed, etc. Control information history, as used herein, refers to information and data about user actions taken within the system. Control information history may include data about actions and about users that took such actions. Control information history may include data and information about control actions taken by a user, e.g., process parameter adjustments, as well as physical actions taken by a user in interacting directly with cell therapy production system 101. Each of the above described data and/or information may be stored as full batch records (i.e., all data pertaining to a particular cell growth batch), collective databases, data extracts (i.e., selected portions of data). Each of the above described data and/or information may be accessed in near-real time by automated process control systems 190 discussed herein.

The automated process control system 190 may be configured as a server (e.g., having one or more server blades, processors, etc.), a personal computer (e.g., a desktop computer, a laptop computer, etc.), a smartphone, a tablet computing device, and/or other device that can be programmed to interface with cell therapy production system 101. In an embodiment, any or all of the functionality of the automated process control system 190 may be performed as part of a cloud computing platform.

The one or more clients may be configured as a personal computer (e.g., a desktop computer, a laptop computer, etc.), a smartphone, a tablet computing device, and/or other device that can be programmed with a user interface for accessing the cell therapy production system 101. In embodiments, the automated process control system 190 and a client may reside within a single system, such as a laptop, desktop, tablet, or other computing device with a user interface.

The network environment represents an example embodiment of an automated process control system 190 configured to control a cell therapy production system 101. Any suitable series of individual or network connections may be employed to permit an automated process control system 190 to cell therapy production system 101 and access required resources such as various data retention systems.

The network may be connected via wired or wireless links. Wired links may include Digital Subscriber Line (DSL), coaxial cable lines, or optical fiber lines. Wireless links may include Bluetooth®, Bluetooth Low Energy (BLE), ANT/ANT+, ZigBee, Z-Wave, Thread, Wi-Fi®, Worldwide Interoperability for Microwave Access (WiMAX®), mobile WiMAX®, WiMAX®-Advanced, NFC, SigFox, LoRa, Random Phase Multiple Access (RPMA), Weightless-N/P/W, an infrared channel or a satellite band. The wireless links may also include any cellular network standards to communicate among mobile devices, including standards that qualify as 2G, 3G, 4G, or 5G. Wireless standards may use various channel access methods, e.g., FDMA, TDMA, CDMA, or SDMA. In some embodiments, different types of data may be transmitted via different links and standards. In other embodiments, the same types of data may be transmitted via different links and standards. Network communications may be conducted via any suitable protocol, including, e.g., http, tcp/ip, udp, ethernet, ATM, etc.

The network may be any type and/or form of network. The geographical scope of the network may vary widely and the network can be a body area network (BAN), a personal area network (PAN), a local-area network (LAN), e.g., Intranet, a metropolitan area network (MAN), a wide area network (WAN), or the Internet. The topology of the network may be of any form and may include, e.g., any of the following: point-to-point, bus, star, ring, mesh, or tree. The network may be of any such network topology as known to those ordinarily skilled in the art capable of supporting the operations described herein. The network may utilize different techniques and layers or stacks of protocols, including, e.g., the Ethernet protocol, the internet protocol suite (TCP/IP), the ATM (Asynchronous Transfer Mode) technique, the SONET (Synchronous Optical Networking) protocol, or the SDH (Synchronous Digital Hierarchy) protocol. The TCP/IP internet protocol suite may include application layer, transport layer, internet layer (including, e.g., IPv4 and IPv4), or the link layer. The network may be a type of broadcast network, a telecommunications network, a data communication network, or a computer network.

The data retention systems may include any type of computer readable storage medium (or media) and/or a computer readable storage device. Such computer readable storage medium or device may be configured to store and provide access to data. Examples of computer readable storage medium or device may include, but is not limited to, an electronic storage device, a magnetic storage device, an optical storage device, an electromagnetic storage device, a semiconductor storage device, or any suitable combination thereof, for example, such as a computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick.

The automated process control system 190 as utilized herein and as described in U.S. Provisional Patent Application No. 62/874,119 includes one or more processors (also interchangeably referred to herein as processors, processor(s), or processor 110 for convenience), one or more storage device(s), and/or other components. In other embodiments, the functionality of the processor may be performed by hardware (e.g., through the use of an application specific integrated circuit (“ASIC”), a programmable gate array (“PGA”), a field programmable gate array (“FPGA”), etc.), or any combination of hardware and software. The storage device includes any type of non-transitory computer readable storage medium (or media) and/or non-transitory computer readable storage device. Such computer readable storage media or devices may store computer readable program instructions for causing a processor to carry out one or more methodologies described here. Examples of the computer readable storage medium or device may include, but is not limited to an electronic storage device, a magnetic storage device, an optical storage device, an electromagnetic storage device, a semiconductor storage device, or any suitable combination thereof, for example, such as a computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, but not limited to only those examples.

The processor is programmed by one or more computer program instructions stored on the storage device. For example, the processor is programmed by an automated process control system (apcs) network manager, an process control manager, an automated process control system (apcs) interface manager, and an automated process control system (apcs) data storage manager. It will be understood that the functionality of the various managers as discussed herein is representative and not limiting. Additionally, the storage device may act as a data retention system to provide data storage. As used herein, for convenience, the various “managers” will be described as performing operations, when, in fact, the managers program the processor (and therefore the automated process control system) perform the operation.

The various components of the automated process control system 190 work in concert to provide control of one or more cell therapy production systems 101 and to provide an interface for a user or other system to interface with one or more cell therapy production systems 101.

In further embodiments, provided herein is a cell therapy production system. Suitably, as shown in FIG. 1, in embodiments, the system is portable. As described herein, such a cell therapy production system 101 suitably includes a blood withdrawal device 110, an automated cell engineering system 300 including an enclosable housing 302. Suitably, a cell separation device 201 is contained within the enclosable housing 302 and fluidly connected to the blood withdrawal device 110. Also included in the system is a cell transduction apparatus 202 contained within the enclosable housing and fluidly connected to the cell separation filter; and suitably a cassette 310 contained within the enclosable housing, the cassette comprising a cell culture chamber 403 fluidly connected to the cell transduction apparatus. The system also suitably further includes a cell therapy infusion device 150 fluidly connected to the cell culture chamber.

As described herein, in embodiments, the cell separation device is a cell separation filter 212 that includes a matrix which captures a T-cell population. Suitably the cell transduction apparatus is an electroporation unit 220.

As described herein, suitably the cassette 310 further comprises one or more fluidics pathways, wherein the fluidics pathways provide recirculation, removal of waste and homogenous gas exchange and distribution of nutrients to the cell culture chamber without disturbing cells within the cell culture chamber. The cassette can also further include one or more of a pH sensor, a glucose sensor, an oxygen sensor, a carbon dioxide sensor, and/or an optical density sensor.

As described herein, the system suitably includes a computer control system and a user interface 304, wherein the user interface is coupled to the computer control system to provide instructions to the automated cell engineering system. In embodiments, the system further includes an automated process control system 190 configured to control the system. In embodiments, the system further includes a bed 180 such that the blood withdrawal device 110 and the cell therapy infusion device 150 are collocated with the bed.

Also provided herein are methods for preparing a cell therapy product. The methods suitably include withdrawing a blood sample from a patient; passing the blood sample through a cell separation device to remove a target cell population from the blood sample; transducing the target cell population with a vector to produce a transduced cell culture; optionally expanding the transduced cell culture; harvesting the cell culture; and infusing the harvested cell culture into the patient. In embodiments, the target cell population is a T-cell population, and suitably expansion of the transduced cell culture (e.g. in a cell culture chamber as described herein) is carried out to generate a sufficient number of T-cells for infusion back into a patient. However, in other embodiments, including embodiments where the target cell population is not a T-cell population, expansion of the cell population may not be required. Instead, the cells can simply be transduced, and then if desired, processed further, prior to infusion into the patient.

As described herein, the cell separation device suitably removes T-cells from the blood sample via a separation filter, and the transducing comprises electroporating the T-cells with a vector including a chimeric antigen receptor. Thus, in embodiments, the systems and methods described herein can be used in the production of chimeric antigen receptor T-cells.

A chimeric antigen receptor T-cell, or “CAR T-cell,” is a T-cell that is modified with a chimeric antigen receptor (CAR) to more specifically target cancer cells. In general, a CAR includes three parts: the ectodomain, the transmembrane domain, and the endodomain. The ectodomain is the region of the receptor that is exposed to extracellular fluid and includes three parts: a signaling peptide, an antigen recognition region, and a spacer. The signaling peptide directs the nascent protein into the endoplasmic reticulum. In CAR, the signaling peptide is a single-chain variable fragment (scFv). The scFv includes a light chain (VL) and a heavy chain (VH) of immunoglobins connected with a short linker peptide. In some embodiments, the linker includes glycine and serine. In some embodiments, the linker includes glutamate and lysine.

The transmembrane domain of the CAR is a hydrophobic α-helix that spans the membrane. In some embodiments, the transmembrane domain of a CAR is a CD28 transmembrane domain. In some embodiments, the CD28 transmembrane domain results in a highly expressed CAR. In some embodiments, the transmembrane domain of a CAR is a CD3-ζ transmembrane domain. In some embodiments, the CD3-ζ transmembrane domain results in a CAR that is incorporated into a native T-cell receptor.

The endodomain of the CAR is generally considered the “functional” end of the receptor. After antigen recognition by the antigen recognition region of the ectodomain, the CARs cluster, and a signal is transmitted to the cell. In some embodiments, the endodomain is a CD3-ζ endodomain, which includes 3 immunoreceptor tyrosine-based activation motifs (ITAMs). In this case, the ITAMs transmit an activation signal to the T-cell after antigen binding, triggering a T-cell immune response.

During production of CAR T-cells, T-cells are removed from a human subject, genetically altered, and re-introduced into a patient to attack the cancer cells. CAR T-cells can be derived from either the patient's own blood (autologous), or derived from another healthy donor (allogenic). In general, CAR T-cells are developed to be specific to the antigen expressed on a tumor that is not expressed in healthy cells.

Methods for producing CAR T-cells utilizing automated cell processing and the COCOON system are described in U.S. Published Patent Application No. 2019/0169572, the disclosure of which is incorporated by reference herein in its entirety.

As described herein, the methods can further include filtering, washing, and/or formulating the harvested cell culture, prior to the infusing. This can occur after expansion of a cell culture if desired or required, or can simply occur directly after transduction. In processes where cell expansion is not needed prior to further processing, the cells can be simply mixed with desired buffers, diluted if desire, formulated, buffered, osmolarity adjusted, and then infused directly into the patient.

In exemplary embodiments, the various elements of the methods described herein can be controlled by an automated process control system. As described herein, the use of an APCS 190, allows for control of the cell therapy production system from a central control system and in embodiments, allows for control of multiple systems. This can include control of systems across a hospital or clinic, across multiple separate locations within a city, a state, a country or even the world, where each separate cell therapy production system is monitored and automatically updated as needed for the various patient feedback and qualities.

### Additional Exemplary Embodiments

Embodiment 1 is a cell therapy production system, comprising: a blood withdrawal device; a cell separation device fluidly connected to the blood withdrawal device; a cell transduction apparatus fluidly connected to the cell separation filter; a cell processing apparatus fluidly connected to the cell transduction apparatus; and a cell therapy infusion device fluidly connected to the cell processing apparatus.

Embodiment 2 includes the system of embodiment 1, wherein the cell separation device is a cell separation filter that includes a matrix which captures a target cell population.

Embodiment 3 includes the system of embodiment 2, wherein the target cell population is a T-cell population.

Embodiment 4 includes the system of any of embodiments 1-3, wherein the cell transduction apparatus is an electroporation unit.

Embodiment 5 includes the system of any of embodiments 1-4, wherein the cell separation filter, the cell transduction apparatus and the cell processing apparatus are contained within an automated cell engineering system.

Embodiment 6 includes the system of any of embodiments 1-5, wherein the cell processing apparatus includes a cell culture chamber.

Embodiment 7 includes the system of any of embodiments 5-6, wherein the automated cell engineering system includes an enclosable housing.

Embodiment 8 includes the system of embodiment 6, further comprising one or more fluidics pathways, wherein the fluidics pathways provide recirculation, removal of waste and homogenous gas exchange and distribution of nutrients to the cell culture chamber without disturbing cells within the cell culture chamber.

Embodiment 9 includes the system of any of embodiments 1-8, further comprising one or more of a pH sensor, a glucose sensor, an oxygen sensor, a carbon dioxide sensor, and/or an optical density sensor.

Embodiment 10 includes the system of any of embodiments 1-9, wherein the system is portable.

Embodiment 11 includes the system of any of embodiments 1-10, further comprising an automated process control system configured to control to the system.

Embodiment 12 includes the system of any of embodiments 1-11, further comprising a bed such that the blood withdrawal device and the cell therapy infusion device are collocated with the bed.

Embodiment 13 is a cell therapy production system, comprising: a blood withdrawal device; an automated cell engineering system, including: an enclosable housing; a cell separation device contained within the enclosable housing and fluidly connected to the blood withdrawal device; a cell transduction apparatus contained within the enclosable housing and fluidly connected to the cell separation filter; and a cassette contained within the enclosable housing, the cassette comprising a cell culture chamber fluidly connected to the cell transduction apparatus; and a cell therapy infusion device fluidly connected to the cell culture chamber.

Embodiment 14 includes the system of embodiment 13, wherein the cell separation device is a cell separation filter includes a matrix which captures a T-cell population.

Embodiment 15 includes the system of embodiment 13 or embodiment 14, wherein the cell transduction apparatus is an electroporation unit.

Embodiment 16 includes the system of any of embodiments 13-15, wherein the cassette further comprises one or more fluidics pathways, wherein the fluidics pathways provide recirculation, removal of waste and homogenous gas exchange and distribution of nutrients to the cell culture chamber without disturbing cells within the cell culture chamber.

Embodiment 17 includes the system of any of embodiments 13-16, wherein the cassette further comprises one or more of a pH sensor, a glucose sensor, an oxygen sensor, a carbon dioxide sensor, and/or an optical density sensor.

Embodiment 18 includes the system of any of embodiments 13-17, further comprising a computer control system and a user interface, wherein the user interface is coupled to the computer control system to provide instructions to the automated cell engineering system.

Embodiment 19 includes the system of any of embodiments 13-18, wherein the system is portable.

Embodiment 20 includes the system of any of embodiments 13-19, further comprising an automated process control system configured to control the system.

Embodiment 21 includes the system of any of embodiments 13-20, further comprising a bed such that the blood withdrawal device and the cell therapy infusion device are collocated with the bed.

Embodiment 22 is a method for preparing a cell therapy product, the method comprising: withdrawing a blood sample from a patient; passing the blood sample through a cell separation device to remove a target T-cell population from the blood sample; transducing the target T-cell population with a vector to produce a transduced cell culture; optionally expanding the transduced cell culture; harvesting the cell culture; and infusing the harvested cell culture into the patient.

Embodiment 23 includes the method of embodiment 22, wherein the cell separation device removes T-cells from the blood sample via a separation filter.

Embodiment 24 includes the method of embodiment 23, wherein the transducing comprises electroporating the T-cells with a vector including a chimeric antigen receptor.

Embodiment 25 includes the method of any of embodiments 22-24, further comprising filtering, washing, and/or formulating the harvested cell culture, prior to the infusing.

Embodiment 26 includes the method of any of embodiments 22-25, wherein (a)-(f) are controlled by an automated process control system.

It will be readily apparent to one of ordinary skill in the relevant arts that other suitable modifications and adaptations to the methods and applications described herein can be made without departing from the scope of any of the embodiments.

It is to be understood that while certain embodiments have been illustrated and described herein, the claims are not to be limited to the specific forms or arrangement of parts described and shown. In the specification, there have been disclosed illustrative embodiments and, although specific terms are employed, they are used in a generic and descriptive sense only and not for purposes of limitation. Modifications and variations of the embodiments are possible in light of the above teachings. It is therefore to be understood that the embodiments may be practiced otherwise than as specifically described.

All publications, patents and patent applications mentioned in this specification are herein incorporated by reference to the same extent as if each individual publication, patent or patent application was specifically and individually indicated to be incorporated by reference.

1. A cell therapy production system, comprising:
(a) a blood withdrawal device;
(b) a cell separation device fluidly connected to the blood withdrawal device;
(c) a cell transduction apparatus fluidly connected to the cell separation filter;
(d) a cell processing apparatus fluidly connected to the cell transduction apparatus; and
(e) a cell therapy infusion device fluidly connected to the cell processing apparatus.

2. The system of claim 1, wherein the cell separation device is a cell separation filter that includes a matrix which captures a target cell population.

3. The system of claim 2, wherein the target cell population is a T-cell population.

4. The system of claim 1, wherein the cell transduction apparatus is an electroporation unit.

5. The system of claim 1, wherein the cell separation filter, the cell transduction apparatus and the cell processing apparatus are contained within an automated cell engineering system.

6. The system of claim 1, wherein the cell processing apparatus includes a cell culture chamber.

7. The system of any one of claim 5, wherein the automated cell engineering system includes an enclosable housing.

8. The system of claim 6, further comprising one or more fluidics pathways, wherein the fluidics pathways provide recirculation, removal of waste and homogenous gas exchange and distribution of nutrients to the cell culture chamber without disturbing cells within the cell culture chamber.

9. The system of claim 8, further comprising one or more of a pH sensor, a glucose sensor, an oxygen sensor, a carbon dioxide sensor, and/or an optical density sensor.

10. The system of claim 1, wherein the system is portable.

11. The system of claim 1, further comprising an automated process control system configured to control to the system.

12. The system of claim 1, further comprising a bed such that the blood withdrawal device and the cell therapy infusion device are collocated with the bed.

13. A cell therapy production system, comprising:
(a) a blood withdrawal device;
(b) an automated cell engineering system, including:
i. an enclosable housing;
ii. a cell separation device contained within the enclosable housing and fluidly connected to the blood withdrawal device;
iii. a cell transduction apparatus contained within the enclosable housing and fluidly connected to the cell separation filter; and
iv. a cassette contained within the enclosable housing, the cassette comprising a cell culture chamber fluidly connected to the cell transduction apparatus; and
(c) a cell therapy infusion device fluidly connected to the cell culture chamber.

14. The system of claim 13, wherein the cell separation device is a cell separation filter includes a matrix which captures a T-cell population.

15. The system of claim 13, wherein the cell transduction apparatus is an electroporation unit.

16. The system of claim 13, wherein the cassette further comprises one or more fluidics pathways, wherein the fluidics pathways provide recirculation, removal of waste and homogenous gas exchange and distribution of nutrients to the cell culture chamber without disturbing cells within the cell culture chamber.

17. The system of claim 13, wherein the cassette further comprises one or more of a pH sensor, a glucose sensor, an oxygen sensor, a carbon dioxide sensor, and/or an optical density sensor.

18. The system of claim 13, further comprising a computer control system and a user interface, wherein the user interface is coupled to the computer control system to provide instructions to the automated cell engineering system.

19. The system of claim 13, wherein the system is portable.

20. The system of claim 13, further comprising an automated process control system configured to control the system.

21. The system of claim 13, further comprising a bed such that the blood withdrawal device and the cell therapy infusion device are collocated with the bed.

22. A method for preparing a cell therapy product, the method comprising:
(a) withdrawing a blood sample from a patient;
(b) passing the blood sample through a cell separation device to remove a target cell population from the blood sample;
(c) transducing the target cell population with a vector to produce a transduced cell culture;
(d) optionally expanding the transduced cell culture;
(e) harvesting the cell culture; and
(f) infusing the harvested cell culture into the patient.

23. The method of claim 22, wherein the cell separation device removes T-cells from the blood sample via a separation filter.

24. The method of claim 23, wherein the transducing comprises electroporating the T-cells with a vector including a chimeric antigen receptor.

25. The method of claim 22, further comprising filtering, washing, and/or formulating the harvested cell culture, prior to the infusing.

26. The method of claim 22, wherein (a)-(f) are controlled by an automated process control system.
